(19) United States
(12) Patent Application Publication
(10) Pub. No.: US 2015/0044263 A1
(43) Pub. Date: Feb. 12, 2015

(54) Kit comprising two separate compositions, notably for cosmetic application

(71) Applicant: CAPSUM, Marseille (FR)

(72) Inventors: Jérôme Bibette, Paris (FR); Sébastien Bardon, Paris (FR); Thomas Delmas, Marseille (FR); Enric Santanach Carreras, Marseille (FR); Mathieu Goutayer, Saint Malo (FR); Marion Courtemanche, Marseille (FR)

(21) Appl. No.: 14/383,148

(22) PCT Filed: Mar. 8, 2013
(86) PCT No.: PCT/EP2013/054785

§ 371 Date: Sep. 5, 2014

(30) Foreign Application Priority Data
Mar. 8, 2012 (FR) 12 52119

(51) Int. Cl. A61K 8/11; A61K 8/73; A61Q 19/00; A61K 8/65

(52) U.S. Cl. CPC A61K 8/11; A61K 8/65; A61K 8/73; A61K 8/733; A61Q 19/00
USPC 424/401

(57) ABSTRACT

A kit comprising two separate compositions (A) and (B). Composition (A) is an aqueous gel with a viscosity of less than 11 Pa·s and contains at least one capsule including: a liquid core with at least one active ingredient and a gelled envelope totally encapsulating the liquid core. The gelled envelope includes at least one polyelectrolyte in the gelled state. Composition (B) includes a depolymerizing agent.

The present invention relates to a kit comprising two separate compositions, notably for cosmetic application.

At the present time, there exist compositions comprising capsules, used in many technical fields. These capsules are generally formed with a core surrounded by a more or less solid envelope.

In the pharmaceutical industry or in the cosmetic industry, the capsules are notably filled with biologically or cosmetically active ingredients. They are notably used for protecting their contents and controlling the release of the active ingredients contained in said capsules.

Such capsules are also used in biochemistry applications for immobilizing cells in bioreactors or as artificial cells in implants.

In the food industry, such capsules are used for containing varied additives which allow improvement in the properties of a food product, such as its taste, or its shelf life.

In all of these applications, the envelopes of the capsules are generally formed with a material which is biocompatible with the human body. These envelopes have to be broken in order to release the active ingredients contained in the capsules. It is therefore necessary that they be sufficiently fragile so that the consumer may easily break the capsules during their use.

However, it may also be advantageous to have a reagent which, in contact with the envelope, triggers a mechanism leading to its failure. Thus, it may be useful to make available to the consumer, capsules both containing a given active ingredient and a reagent allowing the breakage of these capsules, in order that he/she may himself/herself prepare the composition to be used.

The object of the present invention is to provide a system allowing simple and rapid application for the user of active ingredients, in particular present in capsules.

The object of the present invention is also to provide a system allowing a product, notably a cosmetic product to be obtained with satisfactory visual aspect and texture.

Thus, the present invention relates to a kit comprising two separate compositions (A) and (B), wherein:
the composition (A) is an aqueous gel with a viscosity of less than 11 Pa·s, advantageously comprised from 1 to 10 Pa·s, said compositions (A) containing at least one capsule comprising:
a liquid core comprising at least one active ingredient; and
a gelled envelope totally encapsulating the liquid core, said gelled envelope comprising at least one polyelectrolyte in the gelled condition; and
the composition (B) comprises a depolymerising agent.

Within the scope of the present description, by “separate compositions” are meant distinct compositions, for example placed in different compartments. Thus, the compositions (A) and (B) are placed in the same kit, but cannot come into contact in the kit. The compositions (A) and (B) are therefore distinct entities.

### Composition (A)

According to the invention, the kit comprises a composition (A) as an aqueous gel.

According to an embodiment, by <<aqueous gel>> is meant a solution comprising water and a gelling agent.

Preferably, the water mass percentage of the aqueous gel is of at least 70%, notably comprised from 70% to 85%, preferentially comprised from 70% to 80%, based on the total mass of the composition (A).

Within the scope of the present description, by <<gelling agent>>is meant a compound capable of giving a composition the consistency of a gel. By <<gel>>is meant a three-dimensional network of solids diluted in a fluid, which may have properties ranging from soft and ductile to hard and brittle, with the viscosity being typically less than 20 Pa·s, advantageously less than 15 Pa·s, and preferentially less than 10 Pa·s.

The gelling agent is preferably selected from the group consisting of polyosides, galactomannans, polysaccharides, glycosaminoglycans and polyols.

Advantageously, it is selected from the group consisting of xanthan, carrageenan, carob, guar, gellan, hyaluronic acid, glycerol, propanediol or cellulose derivatives.

The aqueous gel is used as a dispersion medium for the capsule and ensures permanent hydration of its gelled envelope.

The viscosity of the aqueous gel is less than 11 Pa·s, advantageously comprised from 1 to 10 Pa·s.

The viscosity of the aqueous gel is preferably greater than 2 Pa·s, preferentially less than 10 Pa·s, advantageously less than 8 Pa·s, for example comprised from 2 Pa·s to 8 Pa·s.

The viscosity of the aqueous gel is preferably comprised from 1 to 10 Pa·s.

The viscosity of the aqueous gel is preferentially comprised from 2 to 10 Pa·s.

The viscosity of the aqueous gel is measured at 25° C.

The viscosity is measured with the following method.

A viscometer of the Brookfield type, typically a digital viscometer Brookfield RVDV-E (torsional torque of the spring of 7187.0 dyne-cm), is used, which is a rotary viscometer with an imposed speed provided with a mobile (spindle). A speed is imposed to the mobile in rotation and the measurement of the exerted torque on the mobile allows determination of the viscosity while knowing the geometry/shape parameters of the mobile used. For example a mobile of size (No.) 05 (Brookfield reference: RV5) is for example used. The shear rate corresponding to the measurement of the viscosity is defined by the mobile used and the speed of rotation of the latter.

The viscosity measurement is carried out over 1 minute at room temperature (T=25° C.+/−2° C.). About 150 g of solution are placed in a beaker with a volume of 250 ml, having a diameter of about 7 cm so that the height of the volume occupied by the 150 g of solution is sufficient for reaching the gauge marked on the mobile. Next, the viscometer is started at a speed of 10 rpm and one waits until the displayed value on the screen is stable. This measurement gives the viscosity of the tested fluid, as mentioned within the scope of the present invention.

Such a viscosity of the aqueous gel allows good suspension of the capsule, notably over a duration of at least one month, at a temperature of 40° C. It also gives the possibility, during the mixing of the composition (A) with the composition (B), of obtaining kinetics for the depolymerization of the envelope of about a few seconds or about thirty minutes or even one hour.

The aqueous gel should not comprise any compound capable of depolymerizing the gelled envelope of the capsule in order to keep the capsule intact until the step for mixing the compositions (A) and (B) of the kit according to the invention.

Advantageously, the aqueous gel is transparent so that the consumer may see the capsule. Its texture is selected depending on the texture which one desires to obtain for the final product, resulting from the mixing of both compositions (A) and (B).

The composition (A) comprises at least one capsule containing a liquid core comprising at least one active ingredient, and a gelled envelope totally encapsulating the liquid core for retaining the liquid core.

Thus, the composition (A) may comprise a single capsule or a plurality of identical or different capsules.

Within the scope of the present description, by <<liquid core>> is meant a liquid internal phase at least partly surrounded with an external phase. The liquid internal phase may notably be in the form of a solution, an emulsion or a suspension.

The liquid core of the capsule comprises at least one active ingredient.

Thus, the liquid core may comprise a single active ingredient or a mixture of several active ingredients.

Within the scope of the present description, by <<active ingredient>> is meant a compound having a beneficial physiological effect on the element on which it acts. For example its purpose is protecting, keeping in good condition, treating, curing, perfuming, aromatizing or coloring.

The active ingredient is advantageously a cosmetic, dermopharmaceutical, pharmaceutical or food agent.

The liquid core may contain the active ingredient as a pure liquid, or as a solution of the active ingredient in a liquid solvent, or as a dispersion such as an emulsion or a suspension of the active ingredient in a liquid.

When the active ingredient is a cosmetic agent, it may be selected from sodium hyaluronate or other moisturizing/repairing molecules, vitamins, enzymes, anti-wrinkle agents, anti-ageing agents, protective/anti-radical agents, anti-oxidants, soothing agents, softening agents, anti-irritation agents, tensing/smoothing agents, emollients, thickeners, anti-orange-peel agents, firming agents, sheathing agents, draining agents, anti-inflammatories, depigmentation agents, bleaches, self-tanners, exfoliants, stimulating cell renewal or stimulating skin microcirculation, UV absorbents or filters, anti-dandruff agents.

A cosmetic agent which may be obtained in the core is for example cited in the 93/35/EEC Directive of the Council dated from Jun. 14, 1993. This product is for example a cream, an emulsion, a lotion, a gel or an oil for the skin (hands, face, feet, etc.), a foundation (liquid, solid), a preparation for baths and showers (salts, foams, oils, gels, etc.), a product for hair care (hair dyes and hair bleaches), a cleansing product (lotions, powders, shampoos), a hair care product (lotions, creams, oils), a hair doing product (lotions, lacquers, brillantines), a shaving product (soaps, foams, lotions, etc.), a product intended to be applied on the lips, a sun care product, a sunless tanning product, a product allowing the skin to be whitened, an anti-wrinkle product.

The dermopharmaceutical agents more particularly designate agents acting at the skin.

When the active ingredient is a pharmaceutical agent, it is advantageously selected from anticoagulants, anti-thrombogenic agents, antimitotics, anti-proliferation agents, anti-adhesion agents, anti-migration agents, cell adhesion promoters, growth factors, anti-parasite molecules, anti-inflammatories, angiogenics, inhibitors of angiogenesis, vitamins, hormones, proteins, anti-fungal agents, anti-microbial molecules, antiseptics or antibiotics.

Alternatively, the liquid core contains reactive agents such as proteins or reagents intended to form a bioreactor, to immobilize cells in bioreactors or to form artificial cells for implants.

The food agents are advantageously vegetable or fruit purees such as mango puree, pear puree, coconut puree, cream of onions, of leeks, of carrots, or other preparations which may mix several fruit or vegetables. Alternatively, these are oils such as food oil, of the olive oil, soybean oil, grape pip oil, sunflower oil type or any other oil extracted from plants, as well as food actives such as probiotics, yeasts, vitamins, minerals or oleo-actives.

Within the scope of the present invention, by a <<gelled envelope>> is meant an external phase at least partly surrounding an internal phase, and comprising a compound in the gel state or as a gel. Preferably, the gelled envelope is an aqueous phase, and typically a hydrogel of the polyelectrolyte in the gelled condition. The gelled envelope may also be designated by the terms of <<membrane>> or <<bark>>.

Preferably, the gelled envelope of the capsule has a thickness of less than 500 μm, advantageously greater than 10 μm.

The gelled envelope is generally formed with a monolayer of a homogenous material.

The gelled envelope of the capsule comprises a gel containing water and a polyelectrolyte advantageously selected from proteins, natural polysaccharides and polyelectrolytes which react to multivalent ions.

By <<polyelectrolyte which reacts to polyvalent ions>>, is meant, in the sense of the present invention, a polyelectrolyte which may pass from a liquid state in an aqueous solution to a gelled state under the effect of contact with a gelling solution containing multivalent ions such as ions of an earth-alkaline metal for example selected from calcium ions, barium ions, magnesium ions.

In the liquid state, the individual chains of polyelectrolyte are substantially free to flow relatively to each other. A 2% by mass aqueous solution of polyelectrolyte then has a purely viscous behavior at the characteristic shearing gradients of the shaping method. The viscosity of this zero-shear solution is between 50 mPa·s and 10,000 mPa·s advantageously between 3,000 mPa·s and 7,000 mPa·s. This viscosity with shear gradients characteristic of flows applied during the manufacturing of the capsules is for example measured by means of a stress or deformation rheometer, imposed at the manufacturing temperature, 25° C. for example. For the measurements, a cone-plane geometry with a diameter comprised from 10 to 50 mm, and a cone angle of at most 2° will be used.

The individual chains of polyelectrolyte in the liquid state advantageously have a molar mass of more than 65,000 g/moles.

In the gelled state, the individual chains of polyelectrolyte form with the multivalent ions, a coherent three-dimensional lattice which retains the liquid core and prevents its flow. The individual chains are retained relatively to each other and cannot freely flow relatively to each other. In this condition, the viscosity of the formed gel is infinite. Further, the gel has a flow stress threshold. This stress threshold is greater than 0.05 Pa. The gel also has a non-zero elastic modulus greater than 35 kPa.

The three-dimensional polyelectrolyte gel contained in the envelope confines the water and the surfactant when it is present. The mass content of the polyelectrolyte in the envelope is for example comprised from 0.5% to 5% based on the total mass of the envelope.

The polyelectrolyte is preferably a harmless biocompatible polymer for the human body. For example, it is produced biologically.

Advantageously, it is selected from polysaccharides, synthetic polyelectrolytes based on acrylates (sodium, lithium, potassium or ammonium polyacrylate or polyacrylamide), synthetic polyelectrolytes based on sulfonates (for example sodium poly(styrene sulfonate)). More particularly, the polyelectrolyte is selected from earth alkaline alginates, such as sodium alginate or potassium alginate, a gellan or a pectin.

According to the embodiment of the invention, the polyelectrolyte is a sodium alginate.

The alginates are produced from brown algae called “laminaria”, designated by the term of “sea weed”.

Such alginates advantageously have an α-L-guluronate content of more than about 50%, preferably more than 55%, or even more than 60%.

The gelled envelope may further contain a surfactant.

The surfactant is advantageously an anionic surfactant, a non-ionic surfactant, a cationic surfactant or a mixture thereof. The molecular weight of the surfactant is comprised between 150 g/mol and 10,000 g/mol, advantageously between 250 g/mol and 1,500 g/mol.

In the case when the surfactant is an anionic surfactant, it is for example selected from alkylsulfates, alkylsulfonates, alkylarylsulfonates, alkaline alkylphosphates, dialkylsulfosuccinates, earth alkaline salts of fatty acids either saturated or not. These surfactants advantageously have at least one hydrophobic hydrocarbon chain having a number of carbons greater than 5, or even 10 and at least one hydrophilic anionic group, such as a sulfate, a sulfonate or a carboxylate group bound to one end of the hydrophobic chain.

In the case when the surfactant is a cationic surfactant, it is for example selected from alkylpyridium or alkylammonium halide salts such as n-ethyldodecylammonium chloride or bromide, cetylammonium chloride or bromide (CTAB). These surfactants advantageously have at least one hydrophobic hydrocarbon chain having a number of carbon atoms greater than 5, or even 10 and at least one hydrophilic cationic group, such as a quaternary ammonium cation.

In the case when the surfactant is a non-ionic surfactant, it is for example selected from polyoxyethylene and/or polyoxypropylene derivatives of fatty alcohols, of fatty acids, or alkylphenols, arylphenols, or from alkylglucosides, polysorbates, cocamides.

According to an embodiment of the invention, the surfactant is sodium laurylsulfate (SLS or SDS).

The mass surfactant content in the envelope is greater than 0.001% and is advantageously greater than 0.1%.

Preferably the capsule has a substantially spherical shape and an outer diameter of more than 0.5 mm, advantageously less than 10 mm and preferentially comprised from 1 to 5 mm. It may also be designated with the term of <<pearl>>.

According to a first embodiment, the capsule is a so-called <<simple>> capsule, meaning that the liquid, viscous or thixotropic core consists of a single internal phase, the internal phase being placed in contact with the gelled envelope. A simple capsule is for example a capsule as described in international application WO 2010/063937 in the name of the applicant.

A simple capsule therefore comprises two distinct phases, a liquid internal phase and an external phase in the gelled state surrounding the internal phase. The active ingredient is then contained in the internal phase.

According to another embodiment, the capsule is a so called <<complex>> capsule, meaning that the liquid, viscous or thixotropic core includes a single intermediate drop of an intermediate phase, the intermediate phase being placed in contact with the gelled envelope, and at least one internal drop of an internal phase being positioned in the intermediate drop.

The liquid core of a complex capsule may thus comprise a continuous intermediate phase within which is found a single drop of internal phase. According to an alternative, the liquid core comprises a continuous intermediate phase within which a plurality of drops of internal phase(s) is found.

The active ingredient of the liquid core may be contained in the intermediate phase and/or in the internal phase of the liquid core.

The complex capsules in particular have a monodispersed distribution. In this respect, the size polydispersity of the complex capsules, measured by the variation coefficient C, consisting of the ratio of the standard deviation over the average, is less than 10%, and notably comprised from 1% to 10%.

This ratio may for example be measured on the basis of diameters measured on at least seven capsules by means of the image processing software package “Image J”, on the basis of a photograph of the capsules taken as a top view with a digital camera.

Alternatively, the mass polydispersity of the capsules may be calculated, on the basis of at least 50 measurements of the mass of 50 complex capsules achieved by means of scales of the Mettler-Toledo type with an accuracy of 0.1 mg.

The liquid core of a complex capsule includes an intermediate drop formed on the basis of an intermediate phase and at least one macroscopic internal drop positioned in the intermediate drop and formed with a substantially non-miscible internal phase with the intermediate phase.

The intermediate phase is for example made on the basis of an aqueous or oily solution.

The flow viscosity of the intermediate phase, measured under the conditions applied during the formation of the complex capsules, is substantially less, for example at least 5% less than the viscosity of the solution intended to form the gelled envelope.

This viscosity with characteristic shear gradients of the flows applied upon manufacturing the capsules is for example measured by means of a stress or deformation rheometer imposed at the manufacturing temperature, 25° C. for example. For the measurements, a cone-plane geometry with a diameter comprised from 10 to 50 mm, and a cone angle of at most 2° will be used.

The intermediate drop of the liquid core is advantageously liquid. In one alternative, the intermediate drop is made on the basis of a thixotropic intermediate phase which is in the liquid state and de-structured when it flows, but which is substantially solid or gelled at rest.

By <<liquid when it flows>>, is meant that the behavior of the intermediate phase is viscous, i.e. the deformation of the material not only depends on the applied stress but also on the duration during which this stress is applied. A way for characterizing this behavior is with a creep test by means of a rheometer on the sample, a characteristic stress of the applied flows during manufacturing is applied and the deformation curve is plotted versus time (data obtained with the software of the rheometer). If the curve has non-zero slope for long times (more than 30 seconds), the intermediate phase may be considered as being liquid. If this slope is zero, the intermediate phase may be considered as being solid.

By <<solid or gelled at rest>>, is meant the behavior of the intermediate solid or gelled phase at rest, i.e. the deformation of the material only depends on the applied stress. A way for characterizing this behavior is with a creep test by means of a rheometer; on the sample, a stress characteristic of those undergone by the capsule at rest versus time is applied (data obtained with the software of the rheometer). If the curve has a zero slope for long times (more than 30 seconds), the intermediate phase may be considered as being solid. If this slope is non-zero, the intermediate phase may be considered as being liquid.

Alternatively, the intermediate drop is gelled. In this case, the intermediate drop is for example formed by gelling a gelling product obtained by a change in temperature, notably by a temperature decrease by at least 10° C. Alternatively, gelling is obtained in the presence of ions, other molecules, or under certain pH or ionic force conditions.

The intermediate drop may comprise one or several cosmetic, dermopharmaceutical, pharmaceutical or food active agents as defined above.

The intermediate drop may also comprise excipients, such as thickeners, or rheology modifiers. These thickeners are for example polymers, cross-polymers, microgels, gums or proteins, including polysaccharides, celluloses, polyosides, polymers and co-polymers based on silicone, colloidal particles (silica, clays, latex . . . ).

The intermediate drop may comprise solid particles and notably mother-of-pearl particles.

Advantageously, the intermediate drop is totally interposed between the internal drop and the gelled envelope. Thus, the totality of the internal surface of the gelled envelope is in contact with the intermediate drop, so that the intermediate drop maintains the internal drop totally away from the gelled envelope.

The capsule advantageously comprises at least two internal drops positioned in the intermediate drop, each internal drop comprises an internal phase, advantageously less than twenty internal drops positioned in the intermediate phase, advantageously less than five internal drops positioned in the intermediate phase, and notably from one to four internal drops positioned in the intermediate phase.

Preferably, the capsule comprises two internal drops positioned in the intermediate phase. Both of these internal drops may notably have internal phases of distinct compositions.

The internal drops are macroscopic. Thus, the maximum transverse dimension of each internal drop, given by its diameter when it is spherical, is greater than 150 μm, and is notably greater than 300 μm. These dimensions are measured by the method using the image processing software package “Image J” as described above.

The minimum volume of at least one internal drop is thus greater than 0.5% of the volume of the core.

The sum of the volumes of said or each internal drop is thus comprised from 0.5% to 65% of the total volume of the core, notably from 1% to 55% of the volume of the core.

Each internal drop advantageously has a spherical shape. Alternatively, the shape of the internal drop is different from a spherical shape, for example an elliptical or lenticular shape.

The internal phase following the internal drops is substantially immiscible with the intermediate phase forming the intermediate drops.

When the intermediate phase is aqueous, the internal phase is oily, and vice versa when the intermediate phase is oily, the internal phase is aqueous.

The viscosity of the internal phase forming the internal drop is for example 10% less than the viscosity of the intermediate phase with the characteristic shear gradients applied during the method for forming the drops, i.e. a viscosity of about 1,000 mPa·s. This viscosity with the characteristic shear gradients of the flows applied during the manufacturing of the capsules is for example measured with a stress or deformation rheometer, imposed at the manufacturing temperature, 25° C. for example. For the measurements, a cone-plane geometry with a diameter comprised from 10 to 50 mm, and a cone angle of at most 2° will be used.

Advantageously, the gelled envelope of the complex capsules is such that the volume ratio Rᵥ of the volume of the liquid core to the volume of the gelled envelope is greater than 2, and is notably greater than 5. This ratio Rᵥ is advantageously less than 50, for example it is comprised between 5 and 10.

Thus, the thickness of the gelled envelope of the complex capsules is notably less than 300 μm, and is for example comprised from 25 to 100 μm.

### Composition (B)

Within the scope of the present invention, the kit comprises a composition (B) comprising a depolymerizing agent.

Within the scope of the present description, by <<depolymerizing agent>> is meant a compound able to embrittle and/or break the gelled envelope of the capsule in order to allow the release of the liquid core in the aqueous gel.

The composition (B) may also be designated by the expression of <<depolymerizing solution>> or <<depolymerizing composition>>.

In the case when the gelled envelope of the capsules of composition (A) contains ion gel particles, the depolymerizing agent is a compound able to chelate the ions which may form the gel of the envelope.

These compounds are able to form highly stable metal complexes. They are able to be bound to metal cations in the form of one of its conjugate bases.

Among these chelating compounds, mention may be made of EDTA or ethylene diamine tetra-acetic acid, EGTA or ethylene glycol tetra-acetic acid, crown ethers such as 1.13-diaza-21-crown-7 or 1.10-diaza-18-crown-6, cryptands, or further acids such as citric acid, acrylic acid, polyacrylic acid, phytic acid, phosphoric acid, tartaric acid, malic acid, or more generally a polyacid, inorganic salts thereof, such as a sodium or potassium salt, or esters thereof.

According to an alternative, the depolymerizing agent is a polymer or a copolymer including at least one monomer having a free acid function after polymerization. The depolymerizing agent is for example selected from polymers or copolymers of acrylic acid, of methacrylic acid, of crotonic acid and of maleic acid, salts thereof or esters thereof.

The depolymerizing agent may also be a salt capable of exchanging with each other the ions which may form the gel of the envelope.

According to an alternative, the depolymerizing agent is selected from monovalent cations of monovalent anions.

According to the embodiment of the invention, the depolymerizing agent is citric acid or one of its salts.

The depolymerizing agent is for example sodium citrate dihydrate.

The mass concentration of the chelating compound is notably greater than 0.5% and is for example comprised from 2% to 30% based on the total mass of the composition (B).

In the case when the gelled envelope of the capsules of composition (A) contains a protein or a natural polysaccharide, the depolymerizing agent is an enzyme capable of degrading the proteins or the polysaccharides.

From among the enzymes capable of degrading the proteins, mention may be made of Aquabeautine®, papain and pepsin.

From among the enzymes capable of degrading polysaccharides, mention may be made of endocellulases, exoglucanases, exoglycosidases which degrade the cellulose fibers by enzymatic hydrolysis.

The contacting and mixing of the compositions (A) and (B) trigger the depolymerization of the gelled envelope of the capsule by the depolymerizing agent. The inter-catenary bonds at the origin of the three-dimensional lattice making up the envelope gradually break. The envelope then passes from the gelled state to the liquid state, thereby releasing the liquid core of the capsule, in the aqueous gel.

The time for mixing the compositions (A) and (B), required for obtaining a homogenous final product, free of residues of capsule envelopes, is directly determined by the kinetics of depolymerization. In order to allow the consumer to obtain the final product within a reasonable time, the mixing time is less than one hour, advantageously comprised from 1 second to 30 mins, and preferentially comprised from 5 seconds to 10 mins.

The mixing time depends on various parameters, notably on the viscosities of the compositions (A) and (B), on the action efficiency of the depolymerizing agent towards the gelled envelope of the capsules, but also on the characteristics of the capsules (size, composition, thickness of the envelope, etc.).

According to an embodiment, the polyelectrolyte of the capsule of the composition (A) is selected from polyelectrolytes which react to calcium ions, such as sodium alginate, and the depolymerizing agent of the composition (B) is selected from calcium chelating agents and salts capable of being exchanged with calcium, such as EDTA.

According to another embodiment, the polyelectrolyte of the capsule of the composition (A) is collagen and the depolymerizing agent is selected from enzymes capable of degrading the proteins, such as Aquabeautine®, papain or pepsin.

Advantageously, the composition (B) of the kit according to the invention has a viscosity comprised from 1 mPa·s to 100 Pa·s, advantageously comprised from 1 mPa·s to 60 Pa·s.

The texture of the composition (B) is selected according to the texture which is desirably obtained for the final product.

The composition (B) is notably in the form of a liquid solution, of a gel, of a cream, of a foam or of an emulsion. Its visual aspect partly determines the visual aspect of the final product.

Advantageously, the composition (B) contains a mass percentage of water of at least 60%, advantageously comprised from 70% to 95%, preferentially comprised from 75% to 95%, based on the total mass of said composition.

In an alternative, the composition (B) further contains one or several active ingredients selected from cosmetic, dermopharmaceutical, pharmaceutical or food agents, as defined above.

The invention also relates to an application of the compositions (A) and (B) of the kit according to the invention.

The invention therefore also relates to the kit as defined above for its use for a simultaneous or separate application over time, notably on the skin, of compositions (A) and (B), as defined above.

The kit of the invention therefore allows application of the compositions (A) and (B) as defined above simultaneously, i.e. together, or one after the other.

According to an embodiment, the compositions (A) and (B) are applied simultaneously. The composition (A) is for example positioned in a first container and the composition (B) in a second container distinct from the first. Both compositions are brought together in a same container, put into contact and mixed. The product obtained by this method is ready to be applied.

More particularly, the consumer for example samples a given amount of the composition (A) and deposits it in a container. He/she then samples a given amount of the composition (B), separate from the composition (A) in the kit, and deposits it in the same container. He/she puts both compositions into contact and mixes them. The obtained product is then ready to be applied, for example on the skin, by the consumer himself/herself. This is therefore a simultaneous application.

Said container may have different shapes; this is for example a cup, a pot, a flask, a tube or a bottle. The mixing may be accomplished by hand, with a spoon, with or without stirring the container for example.

A kit intended for simultaneous application of the compositions is for example a cosmetic kit in which the composition (A) is initially placed in a pot and the composition (B) in a cup or a tube. The consumer pours the composition (B) into the pot where the composition (A) is found and mixes by means of a spoon until a homogenous composition is obtained. He/she may then apply it as a standard cosmetic product.

According to another embodiment, the consumer samples and applies one of the two compositions of the kit, and then samples and applies the other composition in order to mix it with the first and obtain the final product. This is then a separate application over time.

A kit intended for separate application over time of the compositions is for example a cosmetic kit in which the compositions (A) and (B) are placed in two distinct containers. The consumer samples the composition (A) in its container and applies it on the skin. He/she then samples the composition (B) in the other container and also applies it on the skin, at the location where the composition (A) is found. He/she then mixes them with the finger until a homogenous composition is obtained which penetrates into the epidermis for example.

The invention also relates to the use of the kit as described earlier for preparing a cosmetic, dermopharmaceutical, pharmaceutical or food product.

The active ingredient(s) of said product correspond(s) to the active ingredients of the liquid core of the capsule of composition (A) and optionally of composition (B).

The object of the invention is also a method for preparing a cosmetic, dermopharmaceutical, pharmaceutical or food product comprising at least one active ingredient, said method comprising the following steps:
putting into contact the compositions (A) and (B) of the kit according to the invention; and
mixing both compositions (A) and (B).

An object of the invention is more particularly a method for preparing a cosmetic product comprising at least one cosmetic active agent, said method comprising the following steps:
putting into contact the compositions (A) and (B) of the kit according to the invention, said composition (A) comprising at least one cosmetic active agent; and
mixing both compositions (A) and (B).

The products prepared by this method are homogenous compositions without any residues of gelled envelope. They may have very different aspects and textures depending on the compositions (A) and (B) used. It is thus possible to obtain a serum, a cream, a gel or a yogurt for example.

As mentioned above, the mixing time depends on the characteristics of the compositions of the kit and is generally less than one hour, advantageously less than 30 mins, and preferentially less than 10 mins. This mixing time should be reasonable as compared with the use targeted by the kit.

The invention also relates to a method for a non-therapeutic cosmetic treatment of the skin comprising a step for applying on the skin at least one layer of the cosmetic product which may be obtained by the preparation method described earlier.

The invention further relates to the cosmetic use of the cosmetic product which may be obtained by the preparation method described earlier.

The invention also relates to a method for non-therapeutic cosmetic treatment of the skin comprising the following steps:
applying on the skin at least one layer of the composition (A) of the kit according to the invention, and at least one layer of the composition (B) of the kit according to the invention; and
mixing on the skin both applied layers.

The invention will be better understood upon reading the following, only given as an example, and made with reference to the appended drawings, wherein:

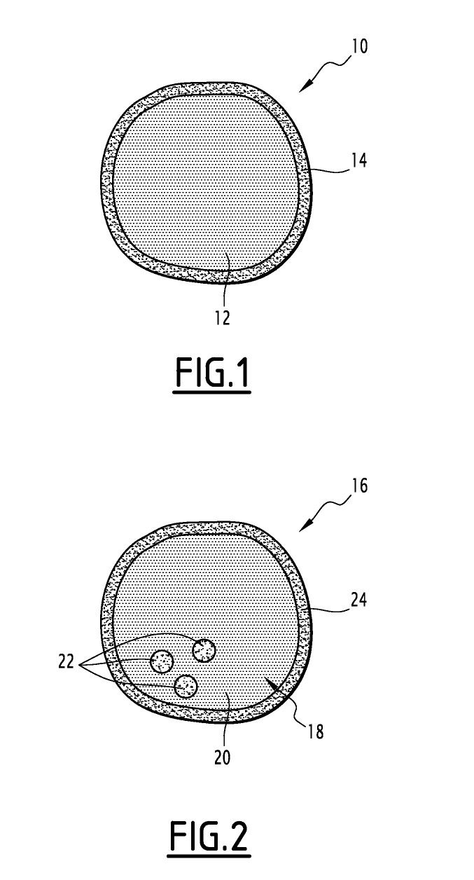

FIG. 1 is a large scale view, as a section along a middle vertical plane of a so-called <<simple>> capsule of the composition (A) of the kit according to the invention; and

FIG. 2 is a large scale view, as a section along a middle vertical plane of a so-called <<complex>> capsule of the composition (A) of the kit according to the invention.

In FIG. 1, a simple capsule 10 of the composition (A) of the kit according to the invention comprises a liquid core 12 consisting of a single internal phase and of a gelled envelope 14 encapsulating the totality of the outer surface of the liquid core 12, for retaining the liquid core 12.

In FIG. 2, a complex capsule 16 of the composition (A) of the kit according to the invention comprises a liquid core 18 which includes an intermediate drop 20 of an intermediate phase, and a plurality of internal drops 22 of an internal phase positioned in the intermediate drop 20. The capsule 16 further comprises a gelled envelope 24 encapsulating the totality of the outer surface of the intermediate drop 20.

### EXAMPLES

### Example 1.1

### Preparation of a Liquid Aqueous Gel (Gel 1)

Table 1 indicates the composition of the gel 1 for which the mass percentage of each constituent is given, based on the total mass of the gel.

| Compound (trade name, supplier) | Mass % |
|---|---|
| Water | 73.7% |
| Glycerol (—, Acros) | 19.0% |
| Propanediol (Zemea, Dupont Tate) | 7.0% |
| Xanthan (Rhodicare T, Rhodia) | 0.3% |

First of all, the xanthan was dispersed in glycols (glycerol and propanediol) with stirring by means of a stirrer with blades. The actives, the preservatives, the perfumes and the coloring agents were then added. Finally, the water was added and the whole was mixed by means of a stirrer with blades for about 1 hour. The gel 1 was thereby obtained.

The gel 1 is transparent. It has a viscosity of 1.5 Pa·s.

The viscosity was measured at 10 rpm by the method using a viscometer of the Brookfield type as described above.

### Example 1.2

### Preparation of a Viscous Aqueous Gel (Gel 2)

Table 2 indicates the composition of the gel 2 for which the mass percentage of each constituent is given, based on the total mass of the gel.

| Compound (trade name, supplier) | Mass % |
|---|---|
| Water | 78.1% |
| Glycerol | 5.0% |
| Propanediol | 10.0% |
| Hyaluronic acid (HMW: 1-2 MDa) | 0.3% |
| (Hasocri, Soliance) | |
| Xanthan | 0.6% |
| Oligogeline (Oligogeline, | 6.0% |
| Biotechmarine) | |
| Ethanol | 2.0% |

First of all the xanthan and the hyaluronic acid was dispersed in glycols (glycerol and propanediol) with stirring by means of a stirrer with blades. The oligogeline was then added and stirring was continued for about 30 mins. The water was finally added and the ethanol and the whole were mixed by means of a stirrer with blades for about 2 hours. The gel 2 was thereby obtained.

The gel 2 is transparent. It has a viscosity of 9.5 Pa·s.

The viscosity was measured at 10 rpm by the method using a viscometer of the Brookfield type described above.

### Example 2.1

### Preparation of Simple Capsules of Liquid Core Comprising an Aqueous Internal Phase (IF) (Series 1)

Table 3 indicates the composition of the capsules of series 1. The mass percentage of each constituent is given, based on the total mass of the phase in which it is found and based on the total mass of a capsule.

| | Trade | Sup- | | Mass | Mass %/ | Mass %/ |
|---|---|---|---|---|---|---|
| | name | plier | INCI | (g) | phase | capsule |
| OF | Water | | Water | 9.81 | 97.86% | 16.34% |
| | Protanal | FMC | Sodium | 0.20 | 2.00% | 0.33% |
| | LF 200S | | alginate | | | |
| | SDS | | Sodium | 0.01 | 0.14% | 0.02% |
| | | | lau- | | | |
| | | | rylsulfate | | | |
| IF | Water | | Water | 47.20 | 94.40% | 78.64% |
| | Hyal- | Soliance | Sodium | 0.50 | 1.00% | 0.84% |
| | uronic | | hyal- | | | |
| | acid | | uronate | | | |
| | Spirulin | Aroma | | 1.25 | 2.50% | 2.08% |
| | | Zone | | | | |
| | Aosaine | Aroma | | 1.05 | 2.10% | 1.75% |
| | | zone | | | | |
| Total | | | | 60.02 | | 100.00% |

A. Preparation of the First Liquid Solution (IF)

944 g of water, 10 g of hyaluronic acid, 25 g of the active spirulin and 21 g of the Aosaine® active were weighed. Hyaluronic acid was added to the water with magnetic stirring. The solution was maintained with stirring for at least 2 hours in order to ensure that it was homogenous. The spirulin and the Aosaine were then added to the solution.

B. Preparation of the Second Liquid Solution (OF)

20 g of sodium alginate, 1,000 g of water and 0.3 g of SDS were weighed. The SDS and the sodium alginate were added to the water with magnetic stirring. The solution was maintained with stirring for at least 24 hours in order to ensure that the alginate was completely dissolved and that the solution is homogenous.

C. Preparation of the Gelling Solution

200 g of calcium chloride were dissolved in 1,000 g of water.

D. Obtaining the Capsules

The method for manufacturing capsules is based on concentric co-extrusion of two solutions, notably described in the international application WO 2010/063937, in order to form double drops.

The capsules of series 1 were therefore obtained according to the following steps:
separate conveyance in a double envelope of the first liquid solution (IF) and of the second liquid solution (OF);
forming at the outlet of the double envelope, a series of drops, each drop comprising a central core formed with a first solution (IF) and a peripheral film formed with a second solution (OF), totally covering the central core;
immersing each drop into a gelling solution so as to have the sodium alginate pass from the second liquid solution (OF) from a liquid state to a gelled state and form the gelled envelope, the central core forming the liquid core;
recovering the formed capsules.

The series 1 of simple capsules was thereby obtained.

### Example 2.2

### Preparation of Complex Capsules of Liquid Core Comprising a Drop of Oily Internal Phase (IF) Suspended in an Intermediate Aqueous Phase (MF) (Series 2)

Table 4 indicates the composition of the capsules of series 2. The mass of each constituent is indicated.

| | Trade name | Supplier | INCI | Mass (g) |
|---|---|---|---|---|
| OF | Water | | Water | 3.80 |
| | Protanal LF 200S | FMC | Sodium alginate | 0.08 |
| | SDS | | Sodium laurylsulfate | 0.01 |
| MF | Water | | Water | 12.73 |
| | Hyaluronic acid | Soliance | Sodium hyaluronate | 0.13 |
| | Rhodicare T | Saci | Xanthan gum | 0.03 |
| | Prestige Magic Gold | Eckart | | 0.10 |
| IF | Phytosqualane | Sophim | Squalane | 5.78 |
| | Omega 3 ceramides | Solabia | | 0.10 |
| | Tea perfume AF B | Azur | Perfume | 0.53 |
| | 26035 | fragrance | | |
| Total | | | | 23.28 |

A. Preparation of the Oily Internal Phase Contained in the First Liquid Solution (IF)

13.5 g of phytosqualane, 0.3 g of the omega 3 ceramide active and 1.2 g of perfume were weighed. The active was added with magnetic stirring to the phytosqualane maintained in a water bath at 70° C. The mixture was maintained with hot stirring until complete dissolution of the active. The solution was then brought back to room temperature. The perfume was then added to the solution.

B. Preparation of the Aqueous Intermediate Phase Contained in the First Liquid Solution (MF)

24.5 g of water, 0.25 g of hyaluronic acid, 0.05 g de Rhodicare T and 0.2 g of Prestige Magic Gold mother-of-pearls were weighed. The hyaluronic acid and Rhodicare were added to the water with magnetic stirring. The solution is maintained with stirring for at least 2 hours in order to ensure that the solution is homogenous. The mothers-of-pearls are then added to the solution.

C. Preparation of the Second Liquid Solution (OF)

20 g of sodium alginate, 1,000 g of water and 0.3 g of SDS were weighed. The SDS and the sodium alginate were added to the water with magnetic stirring. The solution is maintained with stirring for at least 24 hours in order to ensure that the alginate is completely dissolved and that the solution is homogenous.

D. Preparation of the Gelling Solution

200 g of calcium chloride was dissolved in 1,000 g of water.

E. Obtaining the Capsules

The capsules of series 2 were obtained according to the following steps:
injecting the internal phase (IF) through an internal conduit positioned in the double envelope, the internal conduit advantageously opening into and set back from the outlet of the double envelope;
separately conveying in a double envelope a first liquid solution (IF+MF) intended to form the liquid core and a second liquid solution (OF) containing a liquid polyelectrolyte capable of gelling intended to form the gelled envelope;
forming at the outlet of the double envelope a series of liquid bodies including an intermediate phase drop (MF) containing the internal phase (IF) coated with a peripheral film formed with a second solution (OF);
detaching each liquid body away from the double envelope and having each liquid body fall in a volume of air;
immersing each liquid body into a gelling solution containing a reagent capable of reacting with the polyelectrolyte of the film in order to have it pass from a liquid state to a gel state and form the gelled envelope;
recovering the formed capsules.

The series 2 of complex capsules was thereby obtained.

### Example 2.3

### Preparation of Complex Capsules of Liquid Core Comprising an Internal Drop of Aqueous Phase (IF) Suspended in an Oily Intermediate Phase (MF) (Series 3)

Table 5 indicates the composition of the capsules of series 3. The mass percentage of each phase is given, based on the total mass of the capsule and the mass percentage of each constituent is given, based on the total mass of the phase in which it is found and based on the total mass of a capsule.

| | Mass %/ | | | | Mass %/ | Mass % |
|---|---|---|---|---|---|---|
| | capsule | Trade name | Supplier | INCI | phase | /capsule |
| IF | 15.63% | MilliQ Water | — | Water | 98.90% | 15.46% |
| | | Rhodicare T | Rhodia | Xanthan gum | 0.50% | 0.08% |
| | | Allantoine | Aromazone | Allantoin | 0.60% | 0.09% |
| MF | 62.50% | KSG-15 | Shin Etsu | Dimethicone/vinyl | 81.25% | 50.78% |
| | | | | dimethicone | | |
| | | | | Crosspolymer and | | |
| | | | | cyclopentasiloxane | | |
| | | KF-96A-6cs | Shin Etsu | Dimethicone | 18.75% | 11.72% |
| OF | 21.88% | MilliQ Water | — | Water | 94.06% | 20.58% |
| | | Protanal LF | Soliance | Sodium alginate | 2.00% | 0.44% |
| | | 200 FTS | | | | |
| | | Microcare PE | Thor | Phenoxyethanol | 0.80% | 0.18% |
| | | Microcare PTG | Thor | Pentylene glycol | 2.00% | 0.44% |
| | | Timiron | Merck | | 1.00% | 0.22% |
| | | Synwhite 40 | | | | |
| | | SDS | Sigma | Sodium | 0.14% | 0.03% |
| | | | | laurylsulfate | | |

A. Preparation of the Aqueous Internal Phase Contained in the First Liquid Solution (IF)

19.78 g of water, 0.1 g of Rhodicare and 0.12 g of allantoin active were weighed. The Rhodicare® was added to the water with magnetic stirring. The solution is maintained with stirring for at least 2 hours in order to ensure that the solution is homogenous. The active is then added to this solution.

B. Preparation of the Oily Intermediate Phase Contained in the First Liquid Solution (MF)

65 g of KSG-15 and 15 g de KF-96A-6cs were weighed. The KF-96A-6cs was added to the KSG-15 with magnetic stirring.

C. Preparation of the Second Liquid Solution (OF)

0.8 g of sodium alginate, 37.62 g of water, 0.32 g of phenoxyethanol preservative, 0.8 g of pentylene glycol preservative, 0.4 g of Timiron mother-of-pearls and 0.06 g of SDS were weighed. The SDS, the preservatives, the mother-of-pearls and the sodium alginate were added to the water with magnetic stirring. The solution was maintained with stirring for at least 24 hours in order to ensure that the alginate is completely dissolved and that the solution is homogenous.

D. Preparation of the Gelling Solution

200 g of calcium chloride were dissolved in 1,000 g of water.

E. Obtaining the Capsules

The capsules of series 3 were obtained according to the following steps:
injecting the internal phase (IF) through an internal conduit positioned in the double envelope, the internal conduit advantageously, opening into and set back from the outlet of the double envelope;
separately conveying in a double envelope a first liquid solution (IF+MF) intended to form the liquid core and a second liquid solution (OF) containing a liquid polyelectrolyte capable of gelling, intended to form the gelled envelope;
forming at the outlet of the double envelope a series of liquid bodies including an intermediate phase drop (MF) containing the internal phase (IF) coated with a peripheral film formed with a second solution (OF);
detaching each liquid body away from the double envelope and having each liquid body fall in a volume of air;
immersing each liquid body into a gelling solution containing a reagent capable of reacting with the polyelectrolyte of the film in order to have it pass from a liquid state to a gelled state and form the gelled envelope;
recovering the formed capsules.

The series 3 of complex capsules was thereby obtained.

### Example 3.1

### Preparation of Composition (A) No 1

55 g of simple capsules from series 1 (Example 2.1) were added to 45 g of gel 1 (Example 1.1) with planetary gear stirring by means of a three-dimensional Turbulator.

### Example 3.2

### Preparation of the Composition (A) No 2

55 g of complex capsules of series 2 (Example 2.2) were added to 45 g of gel 1 (Example 1.1) with planetary gear stirring by means of a three-dimensional Turbulator.

### Example 3.3

### Preparation of the Composition (A) No 3

55 g of complex capsules of series 3 (Example 2.3) were added to 45 g of gel 1 (Example 1.1) with planetary gear stirring by means of a three-dimensional Turbulator.

### Example 3.4

### Preparation of the Composition (A) No 4

50 g of simple capsules from series 3 (Example 2.3) were added to 50 g of gel 1 (Example 1.1) with planetary gear stirring by means of a three-dimensional Turbulator.

### Example 3.5

### Preparation of the Composition (A) No 5

60 g of complex capsules from series 2 (Example 2.2) were added to 40 g of gel 2 (Example 1.2) with planetary gear stirring by means of a three-dimensional Turbulator.

### Example 3.6

### Preparation of the Composition (A) No 6

40 g of complex capsules from series 2 (Example 2.2) were added to 60 g of gel 2 (Example 1.2) with planetary gear stirring by means of a three-dimensional Turbulator.

### Example 4.1

### Preparation of the Composition (B) No 1

Table 6 indicates the composition (B) no 1 for which the mass percentage of each constituent is given, based on the total mass of the composition.

| Compound (trade name, supplier) | Mass % |
|---|---|
| Water | 80.42% |
| Xanthan | 1.00% |
| Glycerol | 0.13% |
| EDTA | 0.25% |
| Polyacrylate crosspolymer-6 | 1.20% |
| Sodium citrate dihydrate | 6.00% |
| PEG-100 Stearate and glyceryl | 2.00% |
| stearate (Simulsol 165, Seppic) | |
| Shea butter | 2.00% |
| Sweet almond oil | 2.00% |
| Ethylhexyl palmitate | 3.00% |
| Crosslinked methylmethacrylate | 2.00% |
| polymer (Micropearl M 305, Seppic) | |

A. Preparation of the Aqueous Phase
1. The xanthan and the glycerol were first of all weighed. The xanthan was then dispersed in the glycerol, in order to wet it. Preparation 1 was thereby obtained.
2. In parallel, the water and the EDTA were weighed separately, before mixing them with stirring until complete solubilization of the salt. Solution 2 was thereby obtained.
3. Solution 2 was then added to the preparation 1, and the whole was mixed by means of a stirrer with blades until a homogenous dispersion was obtained (about 45 mins). Solution 3 was thereby obtained.
4. The polyacrylate crosspolymer-6 was then added to the solution 3 and the whole was stirred by means of the same stirrer for about one hour. Solution 4 was thereby obtained.
5. Finally, the citrate salts were added to the gelled solution 4 and the mixture was alternately heated to 75° C. and mixed with a blade stirrer.

The actives, the preservatives or further the perfumes and coloring agents may be added at this step.

B. Preparation of the Oily Phase

The sweet almond oil, the ethylhexyl palmitate, the Micropearl and the PEG-100 stearate (oils+emulsifying agents) were successively weighed. The whole was then mixed and heated to 75° C. until a homogenous solution is obtained (about 30 mins).

C. Mixing the 2 Phases

The oily phase and the aqueous phase were then added and the whole was emulsified by means of an Ultra Turrax T25 (IKA Labotechnik) for about 20 to 30 mins.

After decreasing the temperature below 40° C., the whole of the thermosensitive molecules were able to be added, such as the actives, the preservatives or further the perfumes and coloring agents and the stirring was able to be continued until complete dissolution of these molecules in the obtained emulsion. Composition (B) no 1 was thereby obtained.

### Example 4.2

### Preparation of Composition (B) No 2

Table 7 indicates the composition (B) no 2 for which the mass percentage of each constituent is given, based on the total mass of the composition.

| Compound (trade name, supplier) | Mass % |
|---|---|
| Water | 78.15% |
| Disodium EDTA | 0.30% |
| Glycerine 99.0% (glycerol) | 1.00% |
| Xanthan | 0.30% |
| Polyacrylate crosspolymer-6 | 1.50% |
| Homopolymer of acrylic acid, | 0.80% |
| sodium salt (Cosmedia SP, Cognis) | |
| Sodium citrate dihydrate | 6.00% |
| Steareth-2 | 0.50% |
| Steareth-21 | 1.50% |
| Glyceryl stearate and PEG-100 | 1.50% |
| stearate (Simulsol 165, Seppic) | |
| Ethylhexyl palmitate | 4.00% |
| Isohexadecane | 2.00% |
| Stearic acid | 1.00% |
| Ethanol | 1.50% |

A. Preparation of the Aqueous Phase
1. First of all the xanthan and the glycerol were weighed. The xanthan was then dispersed in the glycerol, in order to wet it. Preparation 1 was thereby obtained.
2. In parallel, the water and the EDTA were weighed separately, before mixing them with stirring until complete solubilisation of the salts. Solution 2 was thereby obtained.
3. Solution 2 was then added to the preparation 1, and the whole was mixed by means of a blade stirrer until a homogenous dispersion is obtained (about 45 min). Solution 3 was thereby obtained.
4. The polyacrylate crosspolymer-6 was then added to the solution 3 and the whole was stirred by means of the same stirrer for about 1 hour. Solution 4 was thereby obtained.
5. Finally, the citrate salts were added to the gelled solution 4 and the mixture was alternatively heated to 75° C. and mixed with a blade stirrer.

The actives, the preservatives, or further the perfumes and coloring agents may be added at this step.

B. Preparation of the Oily Phase

The isohexadecane, the Simulsol 165, the ethylhexyl palmitate, the stearic acid and the Steareth 2 and 21 (oils+emulsifying agents) were successively weighed. The whole was then mixed and heated to 75° C. until a homogenous solution is obtained (about 30 min).

C. Mixing the 2 Phases

The oily phase and the aqueous phase was then added and the whole was emulsified by means of an Ultra Turrax T25 (IKA Labotechnik) for about 20 to 30 min.

After decreasing the temperature to below 40° C., the whole of the thermosensitive molecules were able to be added such as the actives, the preservatives, or further the perfumes and coloring agents and stirring was able to be continued until complete solution of these molecules in the obtained emulsion. It is also at this step that the ethanol is added in order to avoid its evaporation at the process temperatures used. Composition (B) no 2 was thereby obtained.

### Example 4.3

### Preparation of Composition (B) No 3

Table 8 indicates the composition (B) no 3 for which the mass percentage of each constituent is given, based on the total mass of the composition.

| Compound (trade name, supplier) | Mass % |
|---|---|
| Water | 93.00% |
| Hydroxyethyl acrylate/sodium acryloyldimethyl | 5.00% |
| taurate copolymer (Sepinov EMT10, Seppic) | |
| Sodium citrate dihydrate | 1.00% |
| Shea olein | 1.00% |

The salts (citric acid and sodium citrate dihydrate) and the water were weighed separately. The salts were then added to the water and the solution was stirred until complete dissolution of all the salts. The actives, the preservatives, or further the perfumes and coloring agents were able to be added at this step.

The acrylate copolymer was then added to the previous solution and the whole was stirred by means of a blade stirrer for about 2 hours before dispersing homogeneously the polymer in the water and obtaining slight gelling of the aqueous solution. Composition (B) no 3 was thereby obtained.

### Example 4.4

### Preparation of the Composition (B) No 4

Table 9 indicates the composition (B) no 4 for which the mass percentage of each constituent is given, based on the total mass of the composition.

| Compound (trade name, supplier) | Mass % |
|---|---|
| Water | 91.70% |
| Hydroxyethyl acrylate/sodium acryloyldimethyl | 2.00% |
| taurate copolymer (Sepinov EMT10, Seppic) | |
| Sodium citrate dehydrate | 5.40% |
| Citric acid | 0.60% |
| EDTA | 0.30% |

The salts (citric acid and sodium citrate dihydrate) and the water were weighed separately. The salts were then added to the water and the solution was stirred until complete dissolution of all the salts. The actives, the preservatives or further the perfumes and coloring agents were able to be added at this step.

The xanthan was then added to the previous solution and the whole was stirred by means of a stirrer blade for about 2 hours in order to homogeneously disperse the polymer in the water and obtain the gelling of the aqueous solution. Composition (B) no 4 was thereby obtained.

### Example 4.5

### Preparation of the Composition (B) No 5

Table 10 indicates the composition (B) no 5 for which the mass percentage of each constituent is given, based on the total mass of the composition.

| Compound (trade name, supplier) | Mass % |
|---|---|
| Water | 80.00% |
| Acrylamide/sodium acrylate copolymer | 10.00% |
| (Massocare TCK A4, Quimica Masso) | |
| Sodium citrate dihydrate | 9.00% |
| Citric acid | 1.00% |

The salts (citric acid and sodium citrate dihydrate) and the water were weighed separately. The salts were then added to the water and the solution was stirred until complete dissolution of all the salts. The actives, the preservatives or further the perfumes and coloring agents were able to be added at this step.

The copolymer of acrylic acid was then added to the previous solution and the whole was stirred by means of a stirrer with blades for about one hour before dispersing the polymer in the water. Composition (B) no 5 was thereby obtained.

### Example 4.6

### Preparation of Composition (B) No 6

Table 11 indicates the composition (B) no 6 for which the mass percentage of each constituent is given, based on the total mass of the composition.

| Compound | Mass % |
|---|---|
| Water | 89.80% |
| Sodium citrate dihydrate | 9.00% |
| Citric acid | 1.00% |
| Xanthan | 0.20% |

The salts (citric acid and sodium citrate dihydrate) and the water were weighed separately. The salts were then added to the water and the solution was stirred until complete dissolution of all the salts. The actives, the preservatives or further the perfumes and coloring agents were able to be added at this step.

The xanthan was then added to the previous solution and the whole was stirred by means of a stirrer with blades for about 2 hours in order to homogeneously disperse the polymer in the water and obtain gelling of the aqueous solution. Composition (B) no 6 was thereby obtained.

### Example 5.1

### Kit for the Preparation and the Use of a Product of the Emulsion Type (Kit 1)

The kit 1 comprises the composition (A) no 1 (Example 3.1) and the composition (B) no 1 (Example 4.1).

The putting into contact and the mixing of the compositions (A) and (B) of the kit 1 gave the possibility of obtaining a product of the emulsion type, comprising 25% by mass of composition (A) and 75% by mass of composition (B), with an opaque aspect and a viscosity equal to 5.5 Pa·s. The depolymerization kinetics are approximately comprised from 2 to 10 min.

The viscosity was measured by the method using a viscometer of the Brookfield type described above.
Use: 15 mL of composition (A) are in a pot, 60 mL of composition (B) are in a flask with a pump.
First possibility: The composition (A) is taken and deposited in the palm of the hand, and the composition (B) will be deposited thereon. The whole is mixed with fingers in order to obtain the final product.
Second possibility: The compositions (A) and (B) are mixed in a cup with a spoon in order to obtain the final product.

### Example 5.2

### Kit for the Preparation and the Use of a Product of the Cream Type (Kit 2)

Kit 2 comprises the composition (A) no 2 (Example 3.2) and the composition (B) no 2 (Example 4.2).

The putting into contact and the mixing of the compositions (A) and (B) of the kit 2 gave the possibility of obtaining a product of the cream type, comprising 20% by mass of composition (A) and 80% by mass of composition (B), with an opaque aspect and a viscosity equal to 38 Pa·s. The depolymerization kinetics are approximately comprised from 2 to 10 min.

The viscosity was measured by the method using a viscometer of the Brookfield type described above.
Use: 40 mL of composition (A) are in a pot and 10 mL of composition (B) are in a cup.

The contents of the cup are poured into the pot and mixed by means of a spoon until a homogenous composition is obtained. It is then applied like a standard cream.

### Example 5.3

### Kit for the Preparation of a Product of the Butter Type (Kit 3)

Kit 3 comprises the composition (A) no 3 (Example 3.3) and the composition (B) no 3 (Example 4.3).

The putting into contact and the mixing of the compositions (A) and (B) of the kit 3 gave the possibility of obtaining a product of the butter type, comprising 15% by mass of composition (A) and 85% by mass of composition (B), with an aspect ranging from being translucent to opaque and with a viscosity equal to 56 Pa·s. The depolymerization kinetics are approximately comprised from 2 to 10 min.

The viscosity was measured by the method using a viscometer of the Brookfield type described above.

### Example 5.4

### Kit for the Preparation of a Product of the Translucent Solution Type (Kit 4)

Kit 4 comprises the composition (A) no 4 (Example 3.4) and the composition (B) no 4 (Example 4.4).

The putting into contact and the mixing of the compositions (A) and (B) of the kit 4 gave the possibility of obtaining a product of the translucent solution type, comprising 18% by mass of composition (A) and 82% by mass of composition (B), with a translucent aspect and a viscosity equal to 0.1 Pa·s. The depolymerization kinetics are approximately comprised from 2 to 10 min.

The viscosity was measured with the method using a viscometer of the Brookfield type described above.

### Example 5.5

### Kit for the Preparation of the Product of the Highly Viscous Gel Type (Kit 5)

Kit 5 comprises the composition (A) no 5 (Example 3.5) and the composition (B) no 5 (Example 4.5).

The putting into contact and the mixing of the compositions (A) and (B) of the kit 5 gave the possibility of obtaining a product of the highly viscous gel type, comprising 50% by mass of composition (A) and 50% by mass of composition (B), with a transparent aspect and a viscosity equal to 26.7 Pa·s. The depolymerization kinetics are approximately comprised from 2 to 10 min.

The viscosity was measured by the method using the viscometer of the Brookfield type described above.

### Example 5.6

### Kit for the Preparation of a Product of the Viscous Gel Type (Kit 6)

Kit 6 comprises composition (A) no 6 (Example 3.6) and the composition (B) no 6 (Example 4.6).

The putting into contact and the mixing of the compositions (A) and (B) of the kit 6 gave the possibility of obtaining a product of the viscous gel type, comprising 10% by mass of composition (A) and 90% by mass of composition (B), with an aspect ranging from being transparent to being translucent and with a viscosity equal to 1 Pa·s. The depolymerization kinetics are approximately comprised from 10 to 30 min.

The viscosity was measured with the method using a viscometer of the Brookfield type described above.

### Example 6

### Influence of Viscosity of the Composition (A)

Compositions (A) of different viscosities were prepared in order to study the influence of viscosity on the physicochemical behavior of the composition (A) and of the capsules. For this, 40% by weight of capsules (those of Example 2.1) are mixed with 60% by weight of aqueous gel. The aqueous gels of different viscosities used fit the following formula, wherein the proportion x of xanthan is variable (typically from 0.1% to 2%) in order to obtain gels with different viscosities:

| Compound (trade name, supplier) | Mass % |
|---|---|
| Water | qsp (100%) |
| Xanthan (Rhodicare T, Rhodia) | x% |
| Phenoxyethanol | 0.8% |
| Pentylene glycol | 2% |
| Glycerol | 8% |
| Propanediol (Zemea, Dupont Tate) | 7% |

The prepared gels have a viscosity varying from 0.5 Pa·s to 10 Pa·s as measured at 25° C., with a speed of 10 rpm with a mobile no 5, according to the method detailed above.

### Example 6.1

### Influence of the Viscosity of the Composition (A) on the Suspensivity of the Capsules

It is preferable according to the invention that the viscosity of the composition (A) allow the capsules to remain in suspension, over a time period of more than 1 month at room temperature.

The capability of variable viscosity compositions (A) of suspending capsules at t₀ and after 1 month at room temperature (between 20 and 25° C.) was evaluated.

The suspensivity of the capsules is evaluated by visual observation. Either the capsules are always uniformly distributed in the gel over the whole height of the flask, and the gel is said to be <<suspensive>> (S), or the capsules are distributed in a non-uniform way in the gel over the height of the flask, and the gel is said to be <<non-suspensive>> (NS). In this second case, the capsules have the tendency of <<creaming>>, thereby moving up to the top of the flask and generating a concentration gradient of capsules, starting from an absence of capsules in the bottom of the flask, to a very high concentration of capsules in contact with each other in the top of the flask.

The results are the following:

| | |
|---|---|
| Viscosity of | Suspensivity of the capsules |
| the gel of (A) | in the gel over time |

| (Pa · s) | t₀ | 1 day | 7 days | 14 days | 31 days |
|---|---|---|---|---|---|
| 0.5 | NS | NS | NS | NS | NS |
| 2 | S | S | S | S | S |
| 5 | S | S | S | S | S |
| 8 | S | S | S | S | S |
| 10 | S | S | S | S | S |
| 12 | S | S | S | S | S |

It is observed that the viscosity of 0.5 Pa·s does not give the possibility of satisfactorily guaranteeing the suspensivity of the capsules in the gel over a period of one month at room temperature.

### Example 6.2

### Influence of the Viscosity of the Composition (A) on the Stability of the Pearls Towards Transport

It is preferable according to the invention that the viscosity of the composition (A) allow the capsules to remain intact during transport of said composition (A).

The impact of shearing on the mechanical stability of the capsules was evaluated in gels of variable viscosity contained in a 10 mL packaging of the dropper type.

8 g of composition (A) are placed in a 10 mL packaging of the dropper type (reference Albea). The dropper, containing the composition (A), is vigorously stirred manually (agitated to and fro).

The state of the capsules is evaluated in terms of deformation of the capsules and of the number of broken capsules according to the following qualitative scale:

| Score | State of the capsules |
|---|---|
| 0 | deformed capsules >10%, breakage >50% |
| 2 | deformed capsules >10%, breakage 20 to 30% |
| 3 | deformed capsules >10%, breakage 10 to 20% |
| 4 | deformed capsules >10%, breakage 5 to 10% |
| 5 | deformed capsules close to 10%, breakage <5% |
| 6 | deformed capsules <10%, breakage <5% |
| 8 | deformed capsules <10%, no breakage |
| 10 | no deformation, no breakage |

The results are the following:

Evaluation of the aspect of the capsules

| Viscosity (Pa · s) | 10 agitations | 25 agitations |
|---|---|---|
| 0.5 | 4 | 3 |
| 1 | 7 | 5 |
| 2 | 8 | 6 |
| 8 | 8 | 7 |
| 10 | 9 | 8 |

It is observed that a viscosity of 0.5 Pa·s does not give the possibility of satisfactorily guaranteeing the stability of the capsules towards transport.

### Example 6.3

### Influence of Viscosity of the Composition (A) on the Depolymerization Kinetics of the Capsules

According to the invention, it is preferable that the viscosity of the composition (A), during its mixing with the depolymerizing composition (B), give the possibility of obtaining an acceptable depolymerization duration and compatible with the use of the cosmetic product obtained by mixing (A) and (B).

The influence of the viscosity of the gel of the composition (A) on the depolymerization duration of the capsules was evaluated.

To 2.5 g of composition (A), a 30% blue citrate solution is added, further comprising 0.2% of xanthan, according to a 1/20 or 1/10 ratio, i.e. 125 μL (ratio 1/20) or 250 μL (ratio 1/10). The mixture is gently homogenized (homogenous blue color) without attempting to shear the capsules.

The depolymerization time of the capsules is evaluated by qualitatively following the state (aspect, shape) of the capsules over time, after mixing the compositions (A) and (B). The homogeneity of the mixture and the presence of envelope residues of the capsules over time are simultaneously followed qualitatively. Both of these parameters are evaluated according to the qualitative scales hereafter:

Homogeneity score

| | |
|---|---|
| 0 (very good) | Homogenous mixture |
| 1 (good) | The remainder of the capsules are still |
| | perceived but they have lost their shape |
| 2 (average) | The capsules begin to be etched by the |
| | depolymerizing solution (B), open capsules |
| | but the envelope still retains the core |
| 4 (poor) | Spherical and non-open capsules |

Score for residues

| | |
|---|---|
| 0 (very good) | No residue |
| 1 (very good) | Residues disappearing from the first application |
| 2 (good) | Residues disappearing after massage (3-5 circular |
| | movements) |
| 3 (average) | Residues disappearing after longer massage (5-10 |
| | circular movements) |
| 5 (poor) | Residues which do not disappear |

The result of the depolymerization is considered as acceptable according to the invention when the score of the homogeneity of the mixture is less than or equal to 2 and those of the membrane residues is less than or equal to 2.

The depolymerization time is estimated to be acceptable according to the invention when it is less than or equal to 15 min. This therefore implies that depolymerization, by its homogeneity and envelope residue aspects, is deemed to be acceptable at 15 min or at shorter times within the scope of the invention.

The results are the following:

| | Evaluation of homogeneity over time | | | | |
|---|---|---|---|---|---|
| Viscosity (Pa · s) | 2 min | 5 min | 15 min | 30 min | 60 min |
| 0.5 | 2 | 0 | 0 | 0 | 0 |
| 1 | 2 | 2 | 1 | 0 | 0 |
| 2 | 3 | 2 | 1 | 1 | 0 |
| 5 | 3 | 3 | 2 | 1 | 0 |
| 8 | 3 | 3 | 2 | 2 | 1 |

| | Evaluation of the residues over time | | | | |
|---|---|---|---|---|---|
| Viscosity (Pa · s) | 2 min | 5 min | 15 min | 30 min | 60 min |
| 0.5 | 1 | 1 | 0 | 0 | 0 |
| 1 | 2 | 1 | 0 | 0 | 0 |
| 2 | 3 | 2 | 1 | 1 | 0 |
| 5 | 3 | 3 | 2 | 1 | 0 |
| 8 | 4 | 3 | 2 | 2 | 1 |

It is observed that up to a viscosity of 8 Pa·s, the result of depolymerization is acceptable after 15 min, both in terms of depolymerization time, of homogeneity of the mixture, and of envelope residues.

### Example 6.4

### Influence of the Viscosity of the Composition (A) on the Texture of the Cosmetic Product Stemming from a (A)+(B) Mixture

It is preferable according to the invention that the viscosity of the composition (A) give the possibility of obtaining a cosmetic product (stemming from the mixture (A)+(B)) having a pleasant texture upon application.

The influence of the viscosity of the gel of the composition (A) on the final texture of the cosmetic product obtained after mixing the compositions (A) and (B) was evaluated.

For this, a composition (B) of the following formula is used, with a measured viscosity of 4.5 Pa·s (at 25° C. according to the method described above):

| Compound (trade name, supplier) | Mass % |
|---|---|
| Water | 79.7% |
| 2-phenoxyethanol (Microcare ® PE) | 0.8% |
| Pentylene glycol (Microcare ® PTG) | 2.0% |
| Trisodium citrate 2-hydrate (PRS-Codex) | 9.0% |
| Anhydrous citric acid (PRS-Codex) | 1.0% |
| Simulgel ™ EPG | 7.5% |

Between 1 and 10 Pa·s, the texture of the mixtures is acceptable according to the invention.

At 13 Pa·s and beyond, the texture of the mixtures is no longer acceptable. Indeed, the increase in the viscosity of the gel leads to obtaining a composition with a tacky and streamlined texture, which takes too much time for penetrating upon application.

1-16. (canceled)

17. A kit comprising two separate compositions (A) and (B), wherein:
the composition (A) is an aqueous gel with a viscosity of less than 11 Pa·s, said composition (A) containing at least one capsule comprising:
a liquid core comprising at least one active ingredient; and
a gelled envelope totally encapsulating the liquid core, said gelled envelope comprising at least one polyelectrolyte in the gelled state; and
the composition (B) comprises a depolymerizing agent.

18. The kit according to claim 17, wherein the composition (A) is an aqueous gel with a viscosity comprised between 1 and 10 Pa·s.

19. The kit according to claim 17, wherein the composition (A) is an aqueous gel with a viscosity comprised from 2 to 10 Pa·s.

20. The kit according to claim 17, wherein the aqueous gel comprises water and a gelling agent.

21. The kit according to claim 20, wherein the mass percentage of water of the aqueous gel is of at least 70% based on the total mass of the composition (A).

22. The kit according to claim 20, wherein the gelling agent of the aqueous gel is selected from the group consisting of polyosides, galactomannans, polysaccharides, glycosaminoglycans and polyols.

23. The kit according to claim 22, wherein the gelling agent of the aqueous gel is selected from the group consisting of xanthan, carrageenan, carob, guar, gellan, hyaluronic acid, glycerol, propanediol or cellulose derivatives.

24. The kit according to claim 17, wherein the capsule has an outer diameter of more than 0.5 mm.

25. The kit according to claim 17, wherein the liquid core of the capsule comprises a single internal drop of an internal phase, placed in contact with the gelled envelope.

26. The kit according to claim 17, wherein the liquid core of the capsule comprises an intermediate drop of an intermediate phase, the intermediate phase being placed in contact with the gelled envelope, and at least one internal drop of an internal phase being positioned in the intermediate drop, the ratio of the volume of the core to the volume of the gelled envelope being greater than 2.

27. The kit according to claim 17, wherein the thickness of the gelled envelope of the capsule of the composition (A) is less than 500 μm.

28. The kit according to claim 17, wherein the polyelectrolyte of the gelled envelope of the capsule is a protein, a natural polysaccharide, or a polyelectrolyte reacting to multivalent ions.

29. The kit according to claim 17, wherein the polyelectrolyte of the gelled envelope of the capsule is collagen.

30. The kit according to claim 17, wherein the polyelectrolyte of the gelled envelope of the capsule is heparan sulfate.

31. The kit according to claim 17, wherein the polyelectrolyte of the gelled envelope of the capsule is a polysaccharide reacting to multivalent ions.

32. The kit according to claim 17, wherein the polyelectrolyte of the gelled envelope of the capsule is selected from the group consisting in alginate, gellan and pectin.

33. The kit according to claim 17 wherein the depolymerizing agent of the composition (B) is selected from calcium chelating agents, salts capable of being exchanged with calcium, enzymes capable of degrading proteins or polysaccharides, and mixtures thereof.

34. A method for non-therapeutic cosmetic treatment of the skin, comprising a step of application of the composition (A) and a step of application of the composition (B), said steps being simultaneous or separate over time.

35. A method for preparing a cosmetic, dermopharmaceutical, pharmaceutical or food product comprising at least one active ingredient, said method comprising the following steps:
putting into contact the compositions (A) and (B) of the kit according to claim 17; and
mixing both compositions (A) and (B).

36. A method for non-therapeutic cosmetic treatment of the skin comprising a step for applying on the skin at least one layer of the cosmetic product which may be obtained by the method according to claim 35.
